(19) United States
(12) Patent Application Publication
(10) Pub. No.: US 2021/0232940 A1
(43) Pub. Date: Jul. 29, 2021

(54) ENCODING AND TRANSMISSION OF KNOWLEDGE, DATA AND RULES FOR EXPLAINABLE AI

(71) Applicant: UMNAI Limited, TA' XBIEX (MT)

(72) Inventors: Angelo DALLI, Floriana (MT); Mauro PIRRONE, Kalkara (MT)

(73) Assignee: UMNAI Limited, TA' XBIEX (MT)

(21) Appl. No.: 17/148,877

(22) Filed: Jan. 14, 2021

Related U.S. Application Data
(60) Provisional application No. 62/964,840, filed on Jan. 23, 2020.

(51) Int. Cl. G06N 5/02; G06N 3/08

(52) U.S. Cl. CPC G06N 5/025; G06N 3/08; G06N 5/022

(57) ABSTRACT

A method for encoding and transmitting knowledge, data and rules, such as for an explainable AI system, may be shown and described. In an exemplary embodiment, the rules may be presented in the disjunctive normal form using first order symbolic logic. Thus, the rules may be machine and human readable, and may be compatible with any known programming language. In an exemplary embodiment, rules may overlap, and a priority function may be assigned to prioritize rules in such an event. The rules may be implemented in a flat or a hierarchical structure. An aggregation function may be used to merge results from multiple rules and a split function may be used to split results from multiple rules. In an exemplary embodiment, rules may be implemented as an explainable neural network (XNN), explainable transducer transformer (XTT), or any other explainable system.

### CROSS-REFERENCE TO RELATED APPLICATIONS

The present patent application claims benefit and priority to U.S. Patent Application No. 62/964,840, filed on Jan. 23, 2020, which is hereby incorporated by reference into the present disclosure.

### FIELD

A method for encoding and transmitting explainable rules for an artificial intelligence system may be shown and described.

### BACKGROUND

Typical neural networks and artificial intelligences (AI) do not provide any explanation for their conclusions or output. An AI may produce a result, but the user will not know how trustworthy that result may be since there is no provided explanation. Modern AIs are black-box systems, meaning that they do not provide any explanation for their output. A user is given no indication as to how the system reached a conclusion, such as what factors are considered and how heavily they are weighed. A result without an explanation could be vague and may not be useful in all cases.

Without intricate knowledge of the inner-workings of the specific AI or neural network being used, a user will not be able to identify what features of the input caused a certain output. Even with an understanding of the field and the specific AI, a user or even a creator of an AI may not be able to decipher the rules of the system since they are often not readable by humans.

Additionally, the rules of typical AI systems are incompatible with applications other than the specific applications they were designed for. They often require high processing power and a large amount of memory to operate and might not be well suited for low-latency applications. There is a need in the field for a human readable and machine adaptable rule format which can allow a user to observe the rules of an AI as it provides an output.

### SUMMARY

According to at least one exemplary embodiment, a method for encoding and transmitting knowledge, data and rules, such as for an explainable AI (XAI) system, may be shown and described. The data may be in machine and human-readable format suitable for transmission and processing by online and offline computing devices, edge and internet of things (IoT) devices, and over telecom networks. The method may result in a multitude of rules and assertions that may have a localization trigger. The answer and explanation may be processed and produced simultaneously. The rules may be applied to domain specific applications, for example by transmitting and encoding the rules, knowledge and data for use in a medical diagnosis imaging scanner system so that it can produce a diagnosis along with an image and explanation of such. The resulting diagnosis can then be further used by other AI systems in an automated pipeline, while retaining human readability and interpretability.

The representation format may consist of a system of disjunctive normal form (DNF) rules or other logical alternatives, like conjunctive normal form (CNF) rules, first-order logic, Boolean logic, second-order logic, propositional logic, predicate logic, modal logic, probabilistic logic, many-valued logic, fuzzy logic, intuitionistic logic, non-monotonic logic, non-reflexive logic, quantum logic, paraconsistent logic and the like. The representation format can also be implemented directly as a hardware circuit, which may be implemented either using flexible architectures like FPGAs or more static architectures like ASICs or analog/digital electronics. The transmission can be affected entirely in hardware when using flexible architectures that can configure themselves dynamically.

The localized trigger may be defined by a localization method, which determines which partition to activate. A partition is a region in the data, which may be disjointing or overlapping. A rule may be a linear or non-linear equation which consists of coefficients with their respective dimension, and the result may represent both the answer to the problem and the explanation coefficients which may be used to generate domain specific explanations that are both machine and human readable. A rule may further represent a justification that explains how the explanation itself was produced. An exemplary embodiment applies an element of human readability to the encoded knowledge, data and rules which are otherwise too complex for an ordinary person to reproduce or comprehend without any automated process.

Explanations may be personalized in such a way that they control the level of detail and personalization presented to the user. The explanation may also be further customized by having a user model that is already known to the system and may depend on a combination of the level of expertise of the user, familiarity with the model domain, the current goals, plans and actions, current session, user and world model, and other relevant information that may be utilized in the personalization of the explanation.

Various methods may be implemented for identifying the rules, such as using an XAI model induction method, eXplainable Neural Networks (XNN), eXplainable artificial intelligence (XAI) models, eXplainable Transducer Transformers (XTT), eXplainable Spiking Nets (XSN), eXplainable Memory Net (XMN), eXplainable Reinforcement Learning (XRL), eXplainable Generative Adversarial Network (XGAN), eXplainable AutoEncoders/Decoder (XAED), eXplainable CNNs (CNN-XNN), Predictive eXplainable XNNs (PR-XNNs), Interpretable Neural Networks (INNs) and related grey-box models which may be a hybrid mix between a black-box and white-box model. Although some examples may reference one or more of these specifically (for example, only XRL or XNN), it may be contemplated that any of the embodiments described herein may be applied to XAIs, XNNs, XTTs, XSNs, INNs, XMNs, XRLs, XGANs, XAEDs, and the like interchangeably. An exemplary embodiment may apply fully to the white-box part of the grey-box model and may apply to at least some portion of the black-box part of the grey-box model. It may be contemplated that any of the embodiments described herein may also be applied to INNs interchangeably.

### BRIEF DESCRIPTION OF THE FIGURES

Advantages of embodiments of the present invention will be apparent from the following detailed description of the exemplary embodiments thereof, which description should be considered in conjunction with the accompanying drawings in which like numerals indicate like elements, in which:

FIG. 1 is an exemplary diagram illustrating a hierarchical rule format.

FIG. 2 is an exemplary schematic flowchart illustrating a white-box model induction method.

FIG. 3 is an exemplary embodiment of a flowchart illustrating the rule-based knowledge embedded in an XNN.

FIG. 4 is an exemplary schematic flowchart illustrating an implementation of an exemplary model induction method.

FIG. 5 is an exemplary schematic flowchart illustrating a method for structuring rules.

FIG. 6 is an exemplary XNN embedded with rule-based knowledge.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein are described in terms of sequences of actions to be performed by, for example, elements of a computing device. It should be recognized by those skilled in the art that the various sequences of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the at least one processor to perform the functionality described herein. Furthermore, the sequence of actions described herein can be embodied in a combination of hardware and software. Thus, the various aspects of the present invention may be embodied in a number of different forms, all of which have been contemplated to be within the scope of the claimed subject matter. In addition, for each of the embodiments described herein, the corresponding form of any such embodiment may be described herein as, for example, “a computer configured to” perform the described action.

An exemplary embodiment presents a method for encoding and transmitting knowledge, data and rules, such as for a white-box AI or neural network, in a machine and human readable manner. The rules or data may be presented in a manner amenable towards automated explanation generation in both online and offline computing systems and a wide variety of hardware devices including but not limited to IoT components, edge devices and sensors, and also amenable to transmission over telecom networks.

An exemplary embodiment results in a multitude of rules and assertions that have a localization trigger together with simultaneous processing for the answer and explanation production, which are then applied to domain specific applications. A localization trigger may be some feature, value, or variable which activates, or triggers, a specific rule or partition. For example, the rules may be transmitted and encoded for use in a medical diagnosis imaging scanner system so that it can produce a diagnosis along with a processed image and an explanation of the diagnosis which can then be further used by other AI systems in an automated pipeline, while retaining human readability and interpretability. Localization triggers can be either non-overlapping for the entire system of rules or overlapping. If they are overlapping, a priority ordering is needed to disambiguate between alternatives and/or alternatively to assign a score or probability value to rank and/or select the rules appropriately.

The representation format may consist of a system of disjunctive normal form (DNF) rules or other logical alternatives, such as conjunctive normal form (CNF) rules, first-order logic assertions, Boolean logic, first order logic, second order logic, propositional logic, predicate logic, modal logic, probabilistic logic, many-valued logic, fuzzy logic, intuitionistic logic, non-monotonic logic, non-reflexive logic, quantum logic, paraconsistent logic and so on. The representation format can also be implemented directly as a hardware circuit, and also may be transmitted in the form of a hardware circuit if required. The representation format may be implemented, for example, by using flexible architectures such as field programmable gate arrays (FPGA) or more static architectures such as application-specific integrated circuits (ASIC) or analogue/digital electronics. The representation format may also be implemented using neuromorphic hardware. Suitable conversion methods that reduce and/or prune the number of rules, together with optimization of rules for performance and/or size also allow for practical implementation to hardware circuits using quantum computers, with the reduced size of rules enabling the complexity of conversion to quantum enabled hardware circuits to be reduced enough to make it a practical and viable implementation method. The transmission can be affected entirely in hardware when using flexible architectures that can configure themselves dynamically.

The rule-based representation format described herein may be applied for a globally interpretable and explainable model. The terms “interpretable” and “explainable” may have different meanings. Interpretability may be a characteristic that may need to be defined in terms of an interpreter. The interpreter may be an agent that interprets the system output or artifacts using a combination of (i) its own knowledge and beliefs; (ii) goal-action plans; (iii) context; and (iv) the world environment. An exemplary interpreter may be a knowledgeable human.

An alternative to a knowledgeable human interpreter may be a suitable automated system, such as an expert system in a narrow domain, which may be able to interpret outputs or artifacts for a limited range of applications. For example, a medical expert system, or some logical equivalent such as an end-to-end machine learning system, may be able to output a valid interpretation of medical results in a specific set of medical application domains.

It may be contemplated that non-human Interpreters may be created in the future that can partially or fully replace the role of a human Interpreter, and/or expand the interpretation capabilities to a wider range of application domains.

There may be two distinct types of interpretability: (i) model interpretability, which measures how interpretable any form of automated or mechanistic model is, together with its sub-components, structure and behavior; and (ii) output interpretability which measures how interpretable the output from any form of automated or mechanistic model is.

Interpretability thus might not be a simple binary characteristic but can be evaluated on a sliding scale ranging from fully interpretable to un-interpretable. Model interpretability may be the interpretability of the underlying embodiment, implementation, and/or process producing the output, while output interpretability may be the interpretability of the output itself or whatever artifact is being examined.

A machine learning system or suitable alternative embodiment may include a number of model components. Model components may be model interpretable if their internal behavior and functioning can be fully understood and correctly predicted, for a subset of possible inputs, by the interpreter. In an embodiment, the behavior and functioning of a model component can be implemented and represented in various ways, such as a state-transition chart, a process flowchart or process description, a Behavioral Model, or some other suitable method. Model components may be output interpretable if their output can be understood and correctly interpreted, for a subset of possible inputs, by the interpreter.

An exemplary machine learning system or suitable alternative embodiment may be (i) globally interpretable if it is fully model interpretable (i.e. all of its components are model interpretable), or (ii) modular interpretable if it is partially model interpretable (i.e. only some of its components are model interpretable). Furthermore, a machine learning system or suitable alternative embodiment, may be locally interpretable if all its output is output interpretable.

A grey-box, which is a hybrid mix of a black-box with white-box characteristics, may have characteristics of a white-box when it comes to the output, but that of a black-box when it comes to its internal behavior or functioning.

A white-box may be a fully model interpretable and output interpretable system which can achieve both local and global explainability. Thus, a fully white-box system may be completely explainable and fully interpretable in terms of both internal function and output.

A black-box may be output interpretable but not model interpretable, and may achieve limited local explainability, making it the least explainable with little to no explainability capabilities and minimal understanding in terms of internal function. A deep learning neural network may be an output interpretable yet model un-interpretable system.

A grey-box may be a partially model interpretable and output interpretable system, and may be partially explainable in terms of internal function and interpretable in terms of output.

The encoded rule-based format may be considered as an exemplary white-box model. It is further contemplated that the encoded rule-based format may be considered as an exemplary interpretable component of an exemplary grey-box model.

The following is an exemplary high-level structure of an encoded rule format, suitable for transmission over telecom networks and for direct conversion to hardware:
If <Localization Trigger> then (<Answer>, <Explanation>)

<Answer> may be of the form:
If <Answer Context> Then <Answer|Equation>

An “else” part in the <Answer> definition is not needed as it may still be logically represented using the appropriate localization triggers, thus facilitating efficient transmission over telecom networks.

<Explanation> may be of the form:
If <Answer Context> Then <Explanation|Equation>

An “else” part in the <Explanation> definition is not needed as it may still be logically represented using the appropriate localization triggers, thus facilitating efficient transmission over telecom networks. The <Explanation Context> may also form part of the encoded rule format, as will be shown later on.

With reference to the exemplary high-level structure of an encoded rule format, an optional justification may be present as part of the system, for example:
If <Localization Trigger> then (<Answer>, <Explanation>, <Justification>)

Where <Justification> may be of the form:
If <Answer Context>, <Explanation Context> Then <Justification|Equation>

In the medical domain, this high-level definition may be applied as follows in order to explain the results of a medical test. <Localization Trigger> may contain a number of conditions which need to be met for the rule to trigger. For example, in a case involving a heart diagnosis, the localization trigger may contain conditions on attributes such as age, sex, type of chest pain, resting blood pressure, serum cholesterol, fasting blood sugar, resting electrocardiographic results, maximum heart rate achieved, and so on. For image-based diagnosis, the localization trigger may be combined with a CNN network in order to apply conditions on the conceptual features modelled by a convolutional network. Such concepts may be high-level features found in X-ray or MRI-scans, which may detect abnormalities or other causes. Using a white-box variant such as a CNN-XNN allows the trigger to be based on both features found in the input data and symbols found in the symbolic representation hierarchy of XNNs, XTTs, XSNs, INNs, XMNs, XRLs, XGANs or XAEDs. Using a causal model variant such as a C-XNN or a C-XTT allows the trigger to be based on causal model features that may go beyond the scope of simple input data features. For example, using a C-XNN or a C-XTT, the localization trigger may contain conditions on both attributes together with intrinsic/endogenous and exogenous causal variables taken from an appropriate Structural Causal Model (SCM) or related Causal Directed Acyclic Graph (DAG) or practical logical equivalent. For example, for a heart diagnosis, a causal variable may take into consideration the treatment being applied for the heart disease condition experienced by the patient.

<Equation> may contain the linear or non-linear model and/or equation related to the triggered localization partition. The equation determines the importance of each feature. The features in such equation may include high-degree polynomials to model non-linear data, or other non-linear transformations including but not limited to polynomial expansion, rotations, dimensional and dimensionless scaling, state-space and phase-space transforms, integer/real/complex/quaternion/octonion transforms, Fourier transforms, Walsh functions, continuous data bucketization, Haar and non-Haar wavelets, generalized L2 functions, fractal-based transforms, Hadamard transforms, Type 1 and Type 2 fuzzy logic, knowledge graph networks, categorical encoding, difference analysis and normalization/standardization of data and conditional features may be applied to an individual partition, prior to the linear fit, to enhance model performance. Each medical feature such as age or resting blood pressure will have a coefficient which is used to determine the importance of that feature. The combination of variables and coefficients may be used to generate explanations in various formats such as text or visual and may also be combined with causal models in order to create more intelligent explanations.

<Answer> is the result of the <Equation>. An answer determines the probability of a disease. In the exemplary medical diagnosis example discussed previously, binary classification may simply return a number from 0 to 1 indicating the probability of a disease or abnormality. In a trivial setting, 0.5 may represent the cut-off point such that when the result is less than 0.5 the medical diagnosis is negative and when the result is greater or equal to 0.5 the result becomes positive, that is, a problem has been detected.

<Answer Context> may be used to personalize the response and explanation to the user. In the exemplary medical application, the Answer Context may be used to determine the level of explanation which needs to be given to the user. For instance, the explanation given to a doctor may be different than that given to the patient. Likewise, the explanation given to a first doctor may be different from that given to a second doctor; for example, the explanation given to a general practitioner or family medicine specialist who has been seeing the patient may have a first set of details, while the explanation given to a specialist doctor to whom the patient has been referred may have a second set of details, which may not wholly overlap with the first set of details.

The <Answer Context> may also have representations and references to causal variables whenever this is appropriate. For this reason, the <Answer Context> may take into consideration the user model and other external factors which may impact the personalization. These external factors may be due to goal-task-action-plan models, question-answering and interactive systems, Reinforcement Learning world and user/agent models and other relevant models which may require personalized or contextual information. Thus, <Answer|Equation> may be personalized through such conditions.

It may be understood that the exact details of how the <Answer|Equation> concept may be personalized may be context-dependent and vary significantly based on the application; for example, in the exemplary medical application discussed above, it may be contemplated to provide a different personalization of the <Answer|Equation> pairing based on the nature of the patient's problem (with different problems resulting in different levels of detail being provided or even different information provided to each), location-specific information such as an average level of skill or understanding of the medical professional (a nurse practitioner may be provided with different information than a general-practice physician, and a specialist may be provided with different information still; likewise, different types of specialists may exist in different countries, depending on the actions of the relevant regulatory bodies) or laws of the location governing what kind of information can or must be disclosed to which parties, or any other relevant information that may be contemplated.

Personalization can occur in a multitude of ways, including either supervised, semi-supervised or unsupervised methods. For supervised methods, a possible embodiment may implement a user model that is specified via appropriate rules incorporating domain specific knowledge about potential users. For example, a system architect may indicate that particular items need to be divulged, while some other items may be assumed to be known. Continuing with the medical domain example, this may represent criteria such as “A Patient needs to know X and Y. Y is potentially a sensitive issue for the Patient to know. A General Practice doctor needs to know X, Y and Z but can be assumed to know Z already. A Cardiac Specialist needs to know X, Y and A, but does not need to know Z. Y should be flagged and emphasized to a Cardiac Specialist, who needs to acknowledge this item in accordance with approved Medical Protocol 123.” For semi-supervised methods, a possible embodiment is to specify the basic priors and set of assumptions and basic rules for a particular domain, and then allow a causal logic engine and/or a logic inference engine to come up with further conclusions that are then added to the rules, possibly after submitting them for human review and approval. For example, if the system has a list of items that a General Practice doctor generally needs to know, like “All General Practice doctors need to know U, V, W, and Z.” and a case specific rule is entered or automatically inferred like “A General Practice doctor needs to know X, Y and Z” the system can automatically infer the “but can be assumed to know Z already” without human input or intervention.

For unsupervised systems, possible embodiments may implement user-feedback models to gather statistics about what parts of an explanation have proved to be useful, and what parts may be omitted. Another possible embodiment may monitor the user interface and user interaction with the explanation to see how much time was spent on a particular part or item of an explanation. Another possible embodiment may quiz or ask the user to re-explain the explanation itself, and see what parts were understood correctly and which parts where not understood or interpreted correctly, which may indicate problems with the explanation itself or that the explanation needs to be expanded for that particular type of user. These possible signals may be used to automatically infer and create new rules for the user model and to build up the user model itself automatically.

For example, if the vast majority of users who are General Practitioner doctors continuously minimize or hide the part of the explanation that explains item Z in the explanation, the system may automatically infer that “All General Practice doctors do not need to be shown Z in detail.” Possible embodiments of rules and user models in all three cases (supervised, semi-supervised and unsupervised) may possibly include knowledge bases, rule engines, expert systems, Bayesian logic networks, and other methods.

Some items may also take into consideration the sensitivity of the user towards receiving such an explanation, or some other form of emotional, classification or receptive flag, which may be known as attribute flags. The attribute flags are stored in the <Context> part of the explanation (<Explanation Context>). For example, some items may be sensitive for a particular user, when dealing with bad news or serious diseases. Some items may need to be flagged for potentially upsetting or graphic content or may have some form of mandated age restriction or some form of legislated flagging that needs to be applied. Another possible use of the attribute flags is to denote the classification rating of a particular item of information, to ensure that potentially confidential information is not advertently released to non-authorized users as part of an explanation.

The explanation generation mechanism can use these attribute flags to customize and personalize the explanation further, for example, by changing the way that certain items are ordered and displayed, and where appropriate may also ask for acknowledgement that a particular item has been read and understood. The <Answer Context> may also have reliability indicators that show the level of confidence in the different items of the Answer, which may be possibly produced by the system that has created the <Answer|Equation> pairs originally, and/or by the system that is evaluating the answer, and/or by some specialized system that judges the reliability and other related factors of the explanation. This information may be stored as part of the <Answer Context> and may provide additional signals that may aid in the interpretation of the answer and its resulting explanation.

<Localization Trigger> may refer to the partition conditions. A localization trigger may filter data according to a specific condition such as “x>10”. The <Explanation> is the equation of the linear or non-linear equation represented in the rule. The rules may be in a generalized format, such as in the disjunctive normal form, or a suitable alternative. The explanation equation may be an equation which receives various data as input, such as the features of an input, weighs the features according to certain predetermined coefficients, and then produces an output. The output could be a classification and may be binary or non-binary. The explanation may be converted to natural language text or some human-readable format. The <Answer> is the result of the <Explanation>, i.e. the result of the equation. <Answer Context> is a conditional statement which may personalize the answer according to some user, goal, or external data. The <Explanation Context> is also a conditional statement which may personalize the explanation according to user, goal, or external data. <Explanation> may be of the form:
If <Answer Context, Explanation Context> Then <Explanation|(Explanation Coefficients, Context Result)>

An else part in the <Explanation> definition may not be needed as it can still be logically represented using the appropriate localization triggers thus facilitating efficient transmission over telecom networks. The Explanation Coefficients may represent the data for generating an explanation by an automated system, such as the coefficients in the equation relied upon in the <Explanation>, and the <Context Result> may represent the answer of that equation.

A Context Result may be a result which has been customized through some user or external context. The Context Result may typically be used to generate a better-quality explanation including related explanations, links to any particular knowledge rules or knowledge references and sources used in the generation of the Answer, the level of expertise of the Answer, and other related contextual information that may be useful for an upstream component or system that will consume the results of an exemplary embodiment and subject it to further processing. Essentially, then, a <Context Result> may operate as a <Answer|Equation> personalized for the system, rather than being personalized for a user, with the <Context Result> form being used in order to ensure that all information is retained for further processing and any necessary further analysis, rather than being lost through simplification or inadvertent omission or deletion. The <Context Result> may also be used in an automated pipeline of systems to pass on information in the chain of automated calls that is needed for further processes downstream in the pipeline, for example, status information, inferred contextual results, and so on.

Typical black-box systems used in the field do not implement any variation of the Explanation Coefficients concept, which represents one of the main differences between the white-box approach illustrated in an exemplary embodiment in contrast with black-box approaches. The <Explanation Coefficient> function or variable can indicate to a user which factors or features of the input led to the conclusion outputted by the model or algorithm. The Explanation Context function can be empty if there is no context surrounding the conclusion. The Answer Context function may also be empty in certain embodiments if not needed.

The context functions (such as <Explanation Context> and <Answer Context>) may personalize the explanation according to user goals, user profile, external events, world model and knowledge, current answer objective and scenario, etc. The <Answer Context> function may differ from the <Explanation Context> function because the same answer may generate different explanations. For example, the explanation to a patient is different than that to a doctor, therefore the explanation context is different, while still having the same answer. Similarly, the answer context may be applicable in order to customize the actual result, irrespective of the explanation. A trivial rule with blank contexts for both Answer Context and Explanation Context will result in a default catch all rule that is always applicable once the appropriate localization trigger turns off.

Referring to the exemplary embodiment involving a medical diagnosis, the answer context and/or explanation context may be implemented such that they contain conditions on the type of user—whether it is a doctor or a patient, both of which would result in a different explanation, hence different goals and context. Other conditions may affect the result, such as national or global diseases which could impact the outcome and may be applicable for an explanation. Conditions on the level of expertise or knowledge may determine if the user if capable of understanding the explanation or if another explanation should be provided. If the user has already seen a similar explanation, a summary of the same explanation may be sufficient.

The <Answer Context> may alter the Answer which is received from the equation. After an answer is calculated, the Answer Context may impact the answer. For example, referring to the medical diagnosis example, the answer may result in a negative reading, however, the <Answer Context> function may be configured to compensate for a certain factor, such as a previous diagnosis. If the patient has been previously diagnosed with a specific problem, and the artificial intelligence network is serving as a second opinion, this may influence the <Answer Context> and may lead to a different result.

The localization method operates in multiple dimensions and may provide an exact, non-overlapping number of partitions. Multi-dimensional partitioning in m-dimensions, may always be localized with conditions of the form:

∀i, i=1, . . . ,m·lᵢ≤dᵢ≤uᵢ

Where lᵢ is the lower bound for dimension i, uᵢ is the upper bound for dimension i, and dᵢ is a conditional value for dimension i. In the trivial case when a dimension is irrelevant, let lᵢ=∞ and uᵢ=∞.

In an exemplary embodiment with overlapping partitions, some form of a priority or disambiguation vector may be implemented. Partitions overlap when a feature or input can trigger more than one rule or partition. A priority vector P can be implemented to provide priority to the partitions. P may have zero to k values, where k denotes the number of partitions. Each element in vector P may denote the level of priority for each partition. The values in vector P may be equal to one another if the partitions are all non-overlapping and do not require a priority ordering. A ranking function may be applied to choose the most relevant rule or be used in some form of probabilistic weighted combination method. In an alternative embodiment, overlapping partitions may also be combined with some aggregation function which merges the results from multiple partitions. The hierarchical partitions may also be subject to one or more iterative optimization steps that may optionally involve merging and splitting of the hierarchical partitions using some suitable aggregation, splitting, or optimization method. A suitable optimization method may seek to find all paths connected topological spaces within the computational data space of the predictor while giving an optimal gauge fixing that minimizes the overall number of partitions.

Some adjustment function may alter the priority vector depending on a query vector Q. The query vector Q may present an optional conditional priority. A conditional priority function ƒ_{cp}(P, Q) gives the adjusted priority vector P_{A} that is used in the localization of the current partition. In case of non-overlapping partitions, the P and P_{A} vectors are simply the unity vector, and f_{q}, becomes a trivial function as the priority is embedded within the partition itself.

Rules may be of the form:
If <Localization Trigger> then <Answer> and <Explanation>

Localization Trigger may be defined by ƒ_{L}(Q, P_{A}), Answer is defined by ƒ_{A}(Q), and Explanation is defined by ƒ_{X}(Q). The adjusted priority vector can be trivially set using the identity function if no adjustment is needed and may be domain and/or application specific.

The <Context Result> controls the level of detail and personalization which is presented to the user. <Context Result> may be represented as a variable and/or function, depending on the use case. <Context Result> may represent an abstract method to integrate personalization and context in the explanations and answers while making it compatible with methods such as Reinforcement Learning that have various different models and contexts as part of their operation.

For example, in the medical diagnosis exemplary embodiment, the Context Result may contain additional information regarding the types of treatments that may be applicable, references to any formally approved medical processes and procedures, and any other relevant information that will aid in the interpretation of the Answer and its context, while simultaneously aiding in the generation of a quality Explanation.

A user model that is already known to the system may be implemented. The user model may depend on a combination of the level of expertise of the user, familiarity with the model domain, the current goals, any goal-plan-action data, current session, user and world model, and other relevant information that may be utilized in the personalization of the explanation. Parts of the explanation may be hidden or displayed or interactively collapsed and expanded for the user to maintain the right level of detail. Additional context may be added depending on the domain and/or application.

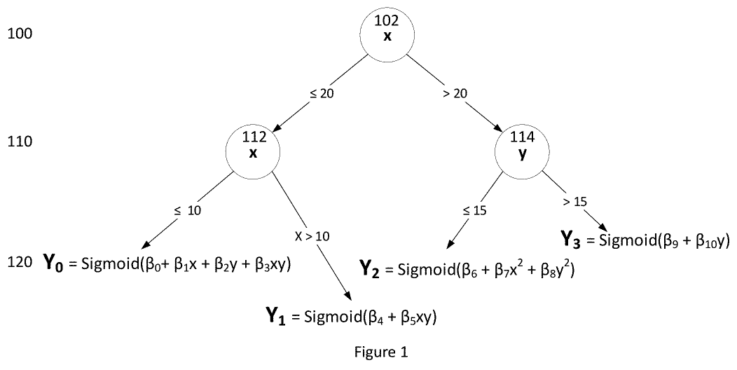

Referring now to exemplary FIG. 1, an exemplary hierarchical partition may be shown. In an exemplary embodiment hierarchical partitions may be represented in a nested or flat rule format.

An exemplary nested rule format may be:

if x ≤ 20:

if x ≤ 10:

Y₀ = Sigmoid(β₀ + β₁x + β₂y + β₃xy)

else:

Y₁ = Sigmoid(β₄ + β₅xy)

else:

if y ≤ 15:

Y₂ = Sigmoid(β₆ + β₇x² + β₈y²)

else:

Y₃ = Sigmoid(β₉ + β₁₀y)

Alternatively, a flat rule format may be implemented. The following flat rule format is logically equivalent to the foregoing nested rule format:

Rule 0
if x≤10:

Y₀=Sigmoid(β₀+β₁x+β₂y+β₃xy)

Rule 1
if x>10 and x≤20:

Y₁=Sigmoid(β₄+β₅xy)

Rule 2
if x>20 and y<15:

Y₂=Sigmoid(β₆+β₇x²+β₈y²)

Rule 3
if x>20 and y>15:

Y₃=Sigmoid(β₉+β₁₀y)

The exemplary hierarchical architecture in FIG. 1 may illustrate a rule with two layers. To illustrate an exemplary implementation of the architecture, let x=24 and y=8. The first layer 100 contains only one rule or partition, where the value of x is analyzed and determines which partition of the second layer 110 to activate. Since x is greater than 20, the second partition 114 of the second layer 110 is activated. The partition 112 of the second layer 110 need not be activated, and the system does not need to expel resources to check whether x≤10 or x>10.

Since the partition 114 was activated, the value of y may be analyzed. Since y≤16, Y₂ may be selected from the answer or value output layer 120. The answer and explanation may describe Y₂, the coefficients within Y₂, and the steps that led to the determination that Y₂ is the appropriate equation. A value may be calculated for Y₂.

Although the previous exemplary embodiment described in FIG. 1 incorporated non-overlapping partitions, in an alternate exemplary embodiment partitions may overlap. In such case, a priority function may be used to determine which partition to activate. Alternatively, an aggregation may also be used to merge results from multiple partitions. Alternatively, a split function may be used to split results from multiple partitions.

For instance, in a different exemplary embodiment with four rules, rules 0-3, when x=30 and y=10 and two rules, rule 1 and rule 2 are triggered such that rule 1 is triggered when x>20 and rule 2 is triggered when x>10 and y≤20. Conditional priority may be required. In this exemplary embodiment, let P={1, 1, 2, 1}, and Q={0, 1, 1, 0}. Some function ƒ_{cp}(P, Q), gives the adjusted priority P_{A}. In this example, P_{A} may be adjusted to {0, 1, 0, 0}. P_{A} may be calculated through a custom function f_{cp}. The output of f_{cp} return P_{A}.

P represents a static priority vector, which is P={1, 1, 2, 1}, and may be hard-coded in the system. Q identifies which rules are triggered by the corresponding input, in this case when x=30 and y=10. In this case, Rules 1 and 2 are triggered.

Rules 0 and 3 do not trigger because their conditions are not met. Within the query vector, Qₖ may represent whether a rule k is triggered. Since Rules 0 and 3 are not triggered, Q₀ and Q₃ are 0, and the triggered Rules 1 and 2 are represented by a 1. Therefore, the query vector becomes Q={0, 1, 1, 0}. The function ƒ_{cp}(P, Q) is a function which takes the vectors P and Q, and returns only 1 active partition which is an adjusted vector. In a trivial exemplary embodiment, ƒ_{cp}(P, Q) may implement one of many contemplated adjustment functions. In this exemplary implementation, ƒ_{cp}(P, Q) simply returns the first hit, resulting in Rule 1 being triggered, rather than Rule 2, since it is ‘hit’ first. Therefore, the adjusted priority (P_{A}) becomes {0, 1, 0, 0}, indicating that Rule 1 will trigger.

When the XAI model encounters time series data, ordered and unordered sequences, lists and other similar types of data, recurrence rules may be referenced. Recurrence rules are rules that may compactly describe a recursive sequence and optionally may describe its evolution and/or change.

The recurrence rules may be represented as part of the recurrent hierarchy and expanded recursively as part of the rule unfolding and interpretation process, i.e. as part of the Answer and Equation components. When the data itself needs to have a recurrence relation to compactly describe the basic sequence of data, the Answer part may contain reference to recurrence relations. For example, time series data produced by some physical process, such as a manufacturing process or sensor monitoring data may require a recurrence relation.

Recurrence relations may reference a subset of past data in the sequence, depending on the type of data being explained. Such answers may also predict the underlying data over time, in both a precise manner, and a probabilistic manner where alternatives are paired with a probability score representing the likelihood of that alternative. An exemplary rule format may be capable of utilizing mathematical representation formats such as Hidden Markov Models, Markov Models, various mathematical series, and the like.

Consider the following ruleset.

$${f}_{r} ( x , y ) = \{ \begin{matrix}Sigm o i d ( {β}_{0} + {β}_{1} x + {β}_{2} y + {β}_{3} x y ) , & x \leq 10 \\ Sigm o i d ( {β}_{4} + {β}_{5} x y ) , & x > 10 ⩓ x \leq 20 \\ Sigm o i d ( {β}_{6} + {β}_{7} {x}^{2} + {β}_{8} {y}^{2} ) , & x > 20 ⩓ y \leq 15 \\ Sigm o i d ( {β}_{9} + {β}_{10} y ) , & x > 20 ⩓ y > 15\end{matrix}$$

These equations may be interpreted to generate explanations. Such explanations may be in the form of text, images, an audiovisual, or any other contemplated form. Explanations may be extracted via the coefficients. In the example above, the coefficients {β₀, . . . , β₁₀} may indicate the importance of each feature. In an example, let x=5 and y=20 in the XAI model function defined by fr(5,20). These values would trigger the first rule, Sigmoid(β₀+β₁x+β₂y+β₃xy) because of the localization trigger “x≤10”. Expanding the equation produces: Sigmoid (β₀+β₁5+β₂20+β100).

From this equation, the multiplication of each coefficient and variable combination may be inputted into a set defined by R={β₁5, β₂20, β100}. β₀, the intercept, may be ignored when analyzing feature importance. By sorting R, the most important coefficient/feature combination may be determined. This “ranking” may be utilized to generate explanations in textual format or in the form of a heatmap for images, or in any other contemplated manner.

The use of the generalized rule format enables a number of additional AI use cases beyond rule-based models, including bias detection, causal analysis, explanation generation, conversion to an explainable neural network, deployment on edge hardware, and integration with expert systems for human-assisted collaborative AI.

An exemplary embodiment provides a summarization technique for simplifying explanations. In the case of high-degree polynomials (2 or higher), simpler features may be extracted. For example, an equation may have the features x, x², y, y², y³, xy with their respective coefficients {θ₁ . . . θ₆}. The resulting feature importance is the ordered set of the elements R={θ₁x, θ₂x², θ₃y, θ₄y², θ₅y³, θ₆xy}. In an exemplary embodiment, elements are grouped irrespective of the polynomial degree for the purposes of feature importance and summarized explanations. In this case, the simplified result set is Rₛ={θ₁x+θ₂x², θ₃y+θ₄y²+θ₅y³, θ₆xy}. Summarization may obtain the simplified ruleset by grouping elements of the equation, irrespective of their polynomial degree. For instance, θ₁ and θ₂ may be grouped together because they are both linked with x, the former with x (degree 1) and the latter x² (degree 2). Therefore, the two are grouped together as θ₁x+θ₂x². Similarly θ₃y and θ₄y² and θ₅y³ are grouped together as θ₃y+θ₄y²+θ₅y³.

A simplified explanation may also include a threshold such that only the top n features are considered, where n is either a static number or percentage value. Other summarization techniques may be utilized on non-linear equations and transformations including but not limited to polynomial expansion, rotations, dimensional and dimensionless scaling, state-space and phase-space transforms, integer/real/complex/quaternion/octonion transforms, Fourier transforms, Walsh functions, continuous data bucketization, Haar and non-Haar wavelets, generalized L2 functions, fractal-based transforms, Hadamard transforms, Type 1 and Type 2 fuzzy logic, knowledge graph networks, categorical encoding, difference analysis and normalization/standardization of data. At a higher level, the multi-dimensional hierarchy of the equations may be used to summarize further. For example, if two summaries can be joined together or somehow grouped together at a higher level, then a high-level summary made up from two or more merged summaries can be created. In extreme cases, all summaries may potentially be merged into one summary covering the entire model. Conversely, summaries and explanations may be split and expanded into more detailed explanations, effectively covering more detailed partitions across multiple summaries and/or explanation parts.

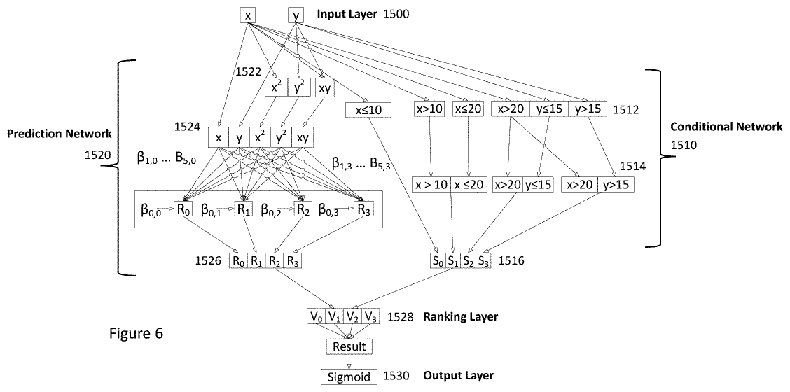

FIG. 6 shows an exemplary embodiment, how the rule-based knowledge described herein may also be embedded into a logically equivalent neural network (XNN). Referring now to exemplary FIG. 6, FIG. 6 may illustrate a schematic diagram of an exemplary high-level XNN architecture. An input layer 1500 may be inputted, possibly simultaneously, into both a conditional network 1510 and a prediction network 1520. The conditional network 1510 may include a conditional layer 1512, an aggregation layer 1514, and a switch output layer (which outputs the conditional values) 1516. The prediction network 1520 may include a feature generation and transformation 1522, a fit layer 1524, and a prediction output layer (value output) 1526. The layers may be analyzed by the selection and ranking layer 1528 that may multiply the switch output by the value output, producing a ranked or aggregated output 1530. The explanations and answers may be concurrently calculated by the XNN by the conditional network and the prediction network. The selection and ranking layer 1528 may ensure that the answers and explanations are correctly matched, ranked, aggregated, and scored appropriately before being sent to the output 1530.

The processing of the conditional network 1510 and the prediction network 1520 is contemplated to be in any order. Depending on the specific application of the XNN, it may be contemplated that some of the components of the conditional network 1510 like components 1512, 1514 and 1516 may be optional or replaced with a trivial implementation. Depending on the specific application of the XNN, it may further be contemplated that some of the components of the prediction network 1520 such as components 1522, 1524 and 1526 may be optional and may also be further merged, split, or replaced with a trivial implementation. The exemplary XNN illustrated in FIG. 6 is logically equivalent to the following system of equations:

$$f ( x , y ) = \{ \begin{matrix}Sigm o i d ( {β}_{0 , 0} + {β}_{1 , 0} x + {β}_{2 , 0} y + {β}_{3 , 0} {x}^{2} + {β}_{4 , 0} {y}^{2} + {β}_{5 , 0} x y ) , & x \leq 10 \\ Sigm o i d ( {β}_{0 , 1} + {β}_{1 , 1} x + {β}_{2 , 1} y + {β}_{3 , 1} {x}^{2} + {β}_{4 , 1} {y}^{2} + {β}_{5 , 1} x y ) , & x > 10 ⩓ x \leq 20 \\ Sigm o i d ( {β}_{0 , 2} + {β}_{1 , 2} x + {β}_{2 , 2} y + {β}_{3 , 2} {x}^{2} + {β}_{4 , 2} {y}^{2} + {β}_{5 , 2} x y ) , & x > 20 ⩓ y \leq 15 \\ Sigm o i d ( {β}_{0 , 3} + {β}_{1 , 3} x + {β}_{2 , 3} y + {β}_{3 , 3} {x}^{2} + {β}_{4 , 3} {y}^{2} + {β}_{5 , 3} x y ) , & x > 20 ⩓ y > 15\end{matrix}$$

A dense network is logically equivalent to a sparse network after zeroing the unused features. Therefore, to convert a sparse XNN to a dense XNN, additional features may be added which are multiplied by coefficient weights of 0. Additionally, to convert from a dense XNN to a sparse XNN, the features with coefficient weights of 0 are removed from the equation.

For example, the dense XNN in FIG. 6 is logically equivalent to the following system of equations:

$$f ( x , y ) = \{ \begin{matrix}Sigm o i d ( {β}_{0 , 0} + {β}_{1 , 0} x + {β}_{2 , 0} y + 0 {x}^{2} + 0 {y}^{2} + {β}_{5 , 0} x y ) , & x \leq 10 \\ Sigm o i d ( {β}_{0 , 1} + 0 x + 0 y + 0 {x}^{2} + 0 {y}^{2} + {β}_{5 , 1} x y ) , & x > 10 ⩓ x \leq 20 \\ Sigm o i d ( {β}_{0 , 2} + 0 x + 0 y + {β}_{3 , 2} {x}^{2} + {β}_{4 , 2} {y}^{2} + 0 x y ) , & x > 20 ⩓ y \leq 15 \\ Sigm o i d ( {β}_{0 , 3} + 0 x + {β}_{2 , 3} y + 0 {x}^{2} + 0 {y}^{2} + 0 x y ) , & x > 20 ⩓ y > 15\end{matrix}$$

Which can be simplified to:

$${f}_{r} ( x , y ) = \{ \begin{matrix}Sigm o i d ( {β}_{0} + {β}_{1} x + {β}_{2} y + {β}_{3} x y ) , & x \leq 10 \\ Sigm o i d ( {β}_{4} + {β}_{5} x y ) , & x > 10 ⩓ x \leq 20 \\ Sigm o i d ( {β}_{6} + {β}_{7} {x}^{2} + {β}_{8} {y}^{2} ) , & x > 20 ⩓ y \leq 15 \\ Sigm o i d ( {β}_{9} + {β}_{10} y ) , & x > 20 ⩓ y > 15\end{matrix}$$

Where β₀=β_{0,0}, β₁=β_{1,0}, β₂=β_{2,0}, β₃=β_{5,0} in rule 0; β₄=β_{0,1}, β₅=β_{5,1} in rule 1; β₆=β_{0,2}, β₇=β_{3,2}, β₈=β_{4,2} in rule 2 and β₉=β_{0,3}, β₁₀=β_{2,3} in rule 3.

The interpretation of the XAI model can be used to generate both human and machine-readable explanations. Human readable explanations can be generated in various formats including natural language text documents, images, diagrams, videos, verbally, and the like. Machine interpretable explanations may be represented using a universal format or any other logically equivalent format. Further, the resulting model may be a white-box AI or machine learning model which accurately captures the original model, which may have been a non-linear black-box model, such as a deep learning or ensemble method. Any model or method that may be queried and that produces a result, such as a classification, regression, or a predictive result may be the source which produces a corresponding white-box explainable model. The source may have any underlying structure, since the inner structure does not need to be analyzed.

An exemplary embodiment may allow direct representation using dedicated, custom built or general-purpose hardware, including direct representation as hardware circuits, for example, implemented using an ASIC, which may provide faster processing time and better performance on both online and offline applications.

Once the XAI model is deployed, it may be suitable for applications where low latency is required, such as real-time or quasi real-time environments. The system may use a space efficient transformation to store the model as compactly as possible using a hierarchical level of detail that zooms in or out as required by the underlying model. As a result, it may be deployed in hardware with low-memory and a small amount of processing power. This may be especially advantageous in various applications. For example, an exemplary embodiment may be implemented in a low power chip for a vehicle. The implementation in the low power chip may be significantly less expensive than a comparable black-box model which requires a higher-powered chip. Further, the rule-based model may be embodied in both software and hardware. Since the extracted model is a complete representation, it may not require any network connectivity or online processing and may operate entirely offline, making it suitable for a practical implementation of offline or edge AI solutions and/or IoT applications.

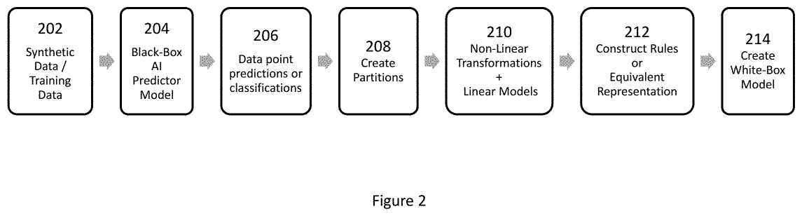

Referring now to exemplary FIG. 2, FIG. 2 may illustrate an exemplary method for extracting rules for an explainable white-box model of a machine learning algorithm from a black-box machine learning algorithm. Since a black-box machine learning algorithm cannot describe or explain its rules, it may be useful to extract those rules such that they may be implemented in a white-box explainable AI or neural network. In an exemplary first step, synthetic or training data may be created or obtained 202. Perturbated variations of the set of data may also be created so that a larger dataset may be obtained without increasing the need for additional data, thus saving resources. The data may then be loaded into the black-box system as an input 204. The black-box system may be a machine learning algorithm of any underlying architecture. In an exemplary embodiment, the machine learning algorithm may be a deep neural network (DNN) and/or a wide neural network (WNN). The black-box system may additionally contain non-linear modelled data. The underlying architecture and structure of the black-box algorithm may not be important since it does not need to be analyzed directly. Instead, the training data may be loaded as input 204, and the output can be recorded as data point predictions or classifications 206. Since a large amount of broad data is loaded as input, the output data point predictions or classifications may provide a global view of the black-box algorithm.

Still referring to exemplary FIG. 2, the method may continue by aggregating the data point predictions or classifications into hierarchical partitions 208. Rule conditions may be obtained from the hierarchical partitions. An external function defined by Partition(X) may identify the partitions. Partition(X) may be a function configured to partition similar data and may be used to create rules. The partitioning function may consist of a clustering algorithm such as k-means, Bayesian, connectivity based, centroid based, distribution based, grid based, density based, fuzzy logic based, entropy, a mutual information (MI) based method, or any other logically suitable methods.

The partition function may also include an ensemble method which would result in a number of overlapping or non-overlapping partitions. The partition function may alternatively include association-based algorithms, causality based partitioning or other logically suitable partitioning implementations.

The hierarchical partitions may organize the output data points in a variety of ways. Data points may contain feature data in various formats including but not limited to 2D or 3D data, such transactional data, sensor data, image data, natural language text, video data, LIDAR data, RADAR, SONAR, and the like. Data points may have one or more associated labels which indicate the output value or classification for a specific data point. Data points may also be organized in a sequence specific manner, such that the order of the data points denote a specific sequence, such as the case with temporal data. In an exemplary embodiment, the data points may be aggregated such that each partition represents a rule or a set of rules. The hierarchical partitions may then be modeled using mathematical transformations and linear models. Although any transformation may be used, an exemplary embodiment may apply a polynomial expansion. Further, a linear fit model may be applied to the partitions 210. Additional functions and transformations may be applied prior to the linear fit depending on the application of the black-box model, such as the softmax or sigmoid function. Other activation functions may also be applicable. The calculated linear models obtained from the partitions may be used to construct rules or some other logically equivalent representation 212. Finally, the rules may be stored in an exemplary rule-based format. Storing the rules as such may allow the extracted model to be applied to any known programming language and may be applied to any computational device. Finally, the rules may be applied to the white-box model 214. The white-box model may store the rules of the black-box model, allowing it to mimic the function of the black-box model while simultaneously providing explanations that the black-box model may not have provided. Further, the extracted white-box model may parallel the original black-box model in performance, efficiency, and accuracy.

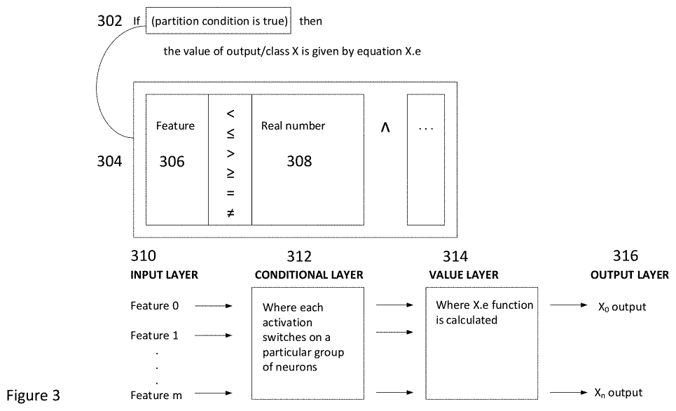

Referring now to exemplary FIG. 3, FIG. 3 may be a schematic flowchart illustrating rule-based knowledge or logically equivalent knowledge embedded in XNNs, XTTs, XSNs, INNs, XMNs, XRLs, XGANs or XAEDs. First, a partition condition 302 may be chosen using a localization method that may reference a number of rules and encoded knowledge. The partition condition may be in any form, such as DNF, CNF, IF-THEN, Fuzzy Logic, and the like. The partition condition may further be defined using a transformation function or combination of transformation functions, including but not limited to polynomial expansion, convolutional filters, fuzzy membership functions, integer/real/complex/quaternion/octonion transforms, Fourier transforms, and others. Partitions can be non-overlapping or overlapping. In the case of non-overlapping partitions, the XNN may take a single path in feed forward mode. In the case of overlapping partitions, the XNN may take multiple paths in feed forward mode and may compute a probability or ranking score for each path. In an exemplary case of an XTT implementation, the XTT will focus its attention depending on the structure of the partitions and effectively compute a probability or ranking score for possible input and output path combinations. In an exemplary case of an XNN, the partition condition 302 can be interpreted as focusing the XNN onto a specific area of the model that is represented. In case of an XTT, the partition condition 302 can be interpreted as additional localization input parameters to the XTT attention model, focusing it onto a specific area of the model that is represented. The partition localization method 304 may be implemented where various features 306 are compared to real numbers 308 repeatedly using CNF, DNF, or any logical equivalent. The localization method values, conditions and underlying equations are selected and identified using an external process, such as an XAI model induction method or a logically equivalent method such as XNNs, XTTs, XSNs, INNs, XMNs, XRLs, XGANs or XAEDs. An XNN may have four main components in its localization or focusing module, which may be part of a conditional network, namely the input layer 310, a conditional layer 312, a value layer 314 and an output layer 316. An XTT may typically implement the input layer 310 as part of its encoders, combine the conditional layer 312 and value layer 314 as part of its attention mechanism, and have the output layer 316 as part of its decoders.

The input layer 310 is structured to receive the various features that need to be processed by the XAI model or equivalent XNNs, XTTs, XSNs, INNs, XMNs, XRLs, XGANs or XAEDs. The input layer 310 feeds the processed features through a conditional layer 312, where each activation switches on a group of neurons. The conditional layer may require a condition to be met before passing along an output. Each condition may be a rule presented in a format as previously described. Further, the input may be additionally analyzed by a value layer 314. The value of the output X (such as in the case of a calculation of an integer or real value, etc.) or the class (such as in the case of a classification application, etc.) X is given by an equation X.e that is calculated by the value layer 314. The X.e function results may be used to produce the output 316. It may be contemplated that the conditional layer and the value layer may occur in any order, or simultaneously.

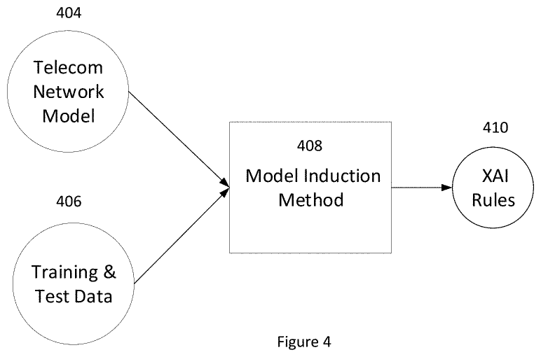

Referring now to exemplary FIG. 4, FIG. 4 may illustrate an exemplary implementation of a model induction method to create rules. Consider an example where XAI rules are used to detect abnormal patterns of data packets within a telecoms network and take appropriate action. Actions may include allowing a user to remain connected, discard part of the data packets or modifying the routing priority of the network to enable faster or slower transmission. For these scenarios, an explanation of why such an action is recommended is generated with an exemplary white-box model, while a black-box would simply recommend the action without any explanation. It would be both useful for the telecoms operator and the customer to understand why the model reached a certain conclusion.

With a white-box model, a user can understand which conditions and features lead to the result. The white-box model may benefit both parties even if they have different goals. From one side, the telecoms operator is interested in minimizing security risk and maximizing network utilization, whereas the customer is interested in uptime and reliability. In one case, a customer may be disconnected on the basis that the current data access pattern is suspicious, and the customer has to close off or remove the application generating such suspicious data patterns before being allowed to reconnect. This explanation helps the customer understand how to rectify their setup to comply for the telecom operator service and helps the telecom operator from losing the customer outright, while still minimizing the risk. Alternatively, the telecom operator may observe that the customer was rejected because of repeated breaches caused by a specific application, which may indicate that there is a high likelihood that the customer may represent an unacceptable security risk within the current parameters of the security policy applied. Further, a third party may also benefit from the explanation: the creator of the telecom security model. The creator of the model may observe that the model is biased such that it over-prioritizes the fast reconnect count variable over other, more important variables, and may alter the model to account for the bias.

The system may account for a variety of factors. Referring to the foregoing telecom example, these factors may include a number of connections in the last hour, bandwidth consumed for both upload and download, connection speed, connect and re-connect count, access point information, access point statistics, operating system information, device information, location information, number of concurrent applications, application usage information, access patterns in the last day, week or month, billing information, and so forth. The factors may each weigh differently, according to the telecom network model.

The resulting answer may be formed by detecting any abnormality and deciding whether a specific connection should be approved or denied. In this case, an equation indicating the probability of connection approval is returned to the user. The coefficients of the equation determine which features impact the probability.

A partition is a cluster that groups data points optionally according to some rule and/or distance similarity function. Each partition may represent a concept, or a distinctive category of data. Partitions that are represented by exactly one rule have a linear model which outputs the value of the prediction or classification. Since the model is linear, the coefficients of the linear model can be used to score the features by their importance. The underlying features may represent a combination of linear and non-linear fits as the rule format handles both linear and non-linear equations.

For example, the following are partitions which may be defined in the telecom network model example.

$$IF Upload\_Bandwidth > 10000 AND Reconnect\_Count <= 3000 THEN Connection\_Approval = \\ …$$

$$IF Upload\_Bandwidth > 1000 \; 0 AND Reconnect\_Count > 3000 THEN Connection\_Approval = \\ …$$

$$IF Bandwidth\_In \_The \_Last \_ 10 \_Minutes >= 500000 THEN Connection\_Approval = …$$

$$IF Device\_Status = “ Idle ” AND Concurrent\_Applications < 10 THEN Connection\_Approval = \\ …$$

The following is an example of the linear model which may be used to predict the Approval probability:

Connection_Approval=Sigmoid(θ₁+θ₂Upload_Bandwidth+θ₃ Reconnect_Count+θ₄Concurrent_Applications+ . . . ).

Each coefficient θᵢ may represent the importance of each feature in determining the final output, where i represents the feature index. The Sigmoid function is being used in this example because it is a binary classification scenario. Another rule may incorporate non-linear transformations such as polynomial expansion, for example θᵢConcurrentApplications² may be one of the features in the rule equation. The creation of rules in an exemplary rule-based format allows the model to not only recommend an option, but also allows the model to explain why a recommendation was made.

Still referring to exemplary FIG. 4, the illustrated system may implement rules to account for a variety of factors. For example, in the illustrated system, these factors may include a number of connections in the last hour, bandwidth consumed for both upload and download, connection speed, connect and re-connect count, access point information, access point statistics, operating system information, device information, location information, number of concurrent applications, application usage information, access patterns in the last day, week or month, billing information, and so forth. The factors may each weigh differently, according to the telecom network model 404. Training and test data 406 may include examples which incorporate various values for the variables, so as to sample a wide range of data. The training and test data 406 may further include synthetically generated data and may also be perturbated. The training and test data 406 may be used as input to the model induction method 408, along with the telecom network model 404. The model induction method 408 may query the telecom network model 404 using the training and test data 406 in order to obtain the rules 410. The obtained rules 410 may be in an exemplary rule-based format, such as DNF, CNF, fuzzy logic, or any other logical equivalent. Such a format allows the rules to be implemented in an explainable system such as an XAI or XNNs, XTTs, XSNs, INNs, XMNs, XRLs, XGANs or XAEDs, since the explainable system could easily read and present the rules to a human user along with an explanation of why a rule was chosen or may apply in a certain scenario.

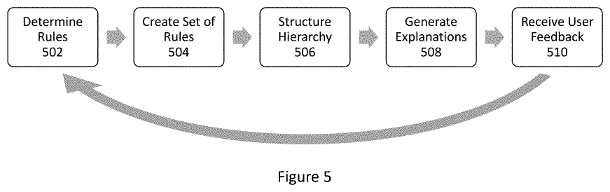

Referring now to exemplary FIG. 5, FIG. 5 may illustrate an exemplary method for structuring rule-based data. In a first step, the initial rules are determined 502. The rules may be determined by a number of methods, such as by the XAI model induction method described in FIG. 2, they may be extracted from an existing XNN, XTT or XAI model, or rules may be determined by any other contemplated method. The determined rules may then be structured in a set 504. The set of rules may be produced by a prediction network and may be a flat set of all possible rules or partitions. In a next optional step, the rules may be structured in a hierarchy 506, as shown in FIG. 1. The hierarchical structure of rules may present further advantages to the system, such as reduced processing time. Next, the system may generate parallel explanations based on how the rules are evaluated 508. The explanations may be processed and displayed parallel to the rules. An optional final step may allow user input to alter the rules 510, and the method may begin again from the initial determination of the rules 502 while incorporating the user input. Since the rules are provided with parallel explanations, a user may be better informed to provide feedback regarding the accuracy or bias of the system.

A further exemplary embodiment utilizes a transform function applied to the output, including the explanation and/or justification output. The transform function may be a pipeline of transformations, including but not limited to polynomial expansions, rotations, dimensional and dimensionless scaling, Fourier transforms, integer/real/complex/quaternion/octonion transforms, Walsh functions, state-space and phase-space transforms, Haar and non-Haar wavelets, generalized L2 functions, fractal-based transforms, Hadamard transforms, Type 1 and Type 2 fuzzy logic, knowledge graph networks, categorical encoding, difference analysis and normalization/standardization of data. The transform function pipeline may further contain transforms that analyze sequences of data that are ordered according to the value of one or more variables, including temporally ordered data sequences. The transform function pipeline may also generate z new features, such that z represents the total number of features by the transformation function. The transformation functions may additionally employ a combination of expansions that are further applied to the output, including the explanation and/or justification output, including but not limited to a series expansion, a polynomial expansion, a power series expansion, a Taylor series expansion, a Maclaurin series expansion, a Laurent series expansion, a Dirichlet series expansion, a Fourier series expansion, a Newtonian series expansion, a Legendre polynomial expansion, a Zernike polynomial expansion, a Stirling series expansion, a Hamiltonian system, Hilbert transform, Riesz transform, a Lyapunov function system, an ordinary differential equation system, a partial differential equation system, and a phase portrait system.

An exemplary embodiment using sequence data and/or temporal data and/or recurrent references, would give partitions and/or rules that may be have references to specific previous values in a specific sequence defined using the appropriate recurrence logic and/or system. In such exemplary embodiment, the following are partitions which may be defined in the telecom network model example.

$$IF Upload\_Bandwidth > 10000 AND Reconnect\_Count [ INTERVAL ( now ( ) - 60 seconds , now ( ) ) ] <= \\ 3000 THEN Connection\_Approval = …$$

$$IF AVERAGE ( Upload\_Bandwidth [ INTERVAL ( current , current - 1000 ] ) > 10000 AND \\ Reconnect\_Count [ now ( ) ] > 3000 THEN Connection\_Approval = … \\ IF Banwidth [ INTERVAL ( now ( ) - 10 minutes , now ( ) ) ] >= 500000 THEN Connection\_Approval = … \\ IF Device\_Status [ INTERVAL ( now ( ) - 10 seconds , now ( ) ] in \{ “ Idle ” \} AND Concurrent\_Applications < 10 THEN Connection\_Approval = …$$

An exemplary embodiment using Fuzzy rules, as herein exemplified using the Mamdani Fuzzy Inference System (Mamdani FIS), would give partitions and/or rules that may be defined using fuzzy sets and fuzzy logic. In such exemplary embodiment, the following are partitions which may be defined in the telecom network model example.

$$IF Upload\_Bandwidth is high AND Reconnect\_Count is low THEN Connection\_Approval = …$$

$$IF Upload\_Bandwidth is high AND Reconnect\_Count is medium THEN Connection\_Approval = \\ …$$

$$IF Banwidth\_In \_The \_Last \_ 10 \_Minutes is very\_high THEN Connection\_Approval = …$$

$$IF Device\_Status = “ Idle ” AND Concurrent\_Applications is low THEN Connection\_Approval = \\ …$$

It is further contemplated that in such exemplary embodiment, other types of fuzzy logic systems, such as the Sugeno Fuzzy Inference System (Sugeno FIS) may be utilized. The main difference in such an implementation choice is that the Mamdani FIS guarantees that the resulting explainable system is fully white-box, while the utilization of a Sugeno FIS may result in a grey-box system.

An exemplary rule-based format may provide several advantages. First, it allows a wide variety of knowledge representation formats to be implemented with new or existing AI or neural networks and is compatible with all known machine learning systems. Further, the rule-based format may be edited by humans and machines alike since it is easy to understand and comprehensible and still compatible with any programming language. An exemplary rule may be represented using first order symbolic logic, such that it may interface with any known programming language or computing device. In an exemplary embodiment, explanations may be generated via multiple methods and translated into a universal manner for use in an embodiment. Both global and local explanations can be produced.

Additionally, an exemplary rule format may form the foundation of an XAI, XNN, XTT, INN, XSN, XMN, XRL, XGAN, XAED system or suitable logically equivalent white-box or grey-box explainable machine learning system. It is further contemplated that an exemplary rule format may form the foundation of causal logic extraction methods, human knowledge incorporation and adjustment/feedback techniques, and may be a key building block for collaborative intelligence AI methods. The underlying explanations may be amenable to domain independent explanations which may be transformed into various types of machine and human readable explanations, such as text, images, diagrams, videos, and the like.

An exemplary embodiment in an Explanation and Interpretation Generation System (EIGS) utilizes an implementation of the exemplary rule format to serve as a practical solution for the transmission, encoding and interchange of results, explanations, justifications and EIGS related information.

In an exemplary embodiment, the encoding of the XAI model may be encoded as a set of rules, an XNN, XTT, explainable spiking network (XSN), explainable memory network (XMN), explainable reinforcement learning agent (XRL), explainable generative adversarial network (XGAN), explainable autoencoder/decoder (XAED), or any other explainable system.

Transmission of an exemplary XAI model is achieved by saving the contents of the model, which may include partition data, coefficients, transformation functions and mappings, and the like. Transmission may be done, for example, offline on an embedded hardware device or online using cloud storage systems for saving the contents of the XAI model. XAI models may also be cached in memory for fast and efficient access. When transmitting and processing XAI models, a workflow engine or pipeline engine may be used such that it takes some input, transforms it, executes one or more XAI models and applies further post-hoc processing on the result of the XAI model. Transmission of data may also generate data for subsequent processes, including but not limited to other XAI workflows or XAI models.

An exemplary rule format may be embodied in both software and hardware and may not require a network connection or online processing, and thus may be amenable to edge computing techniques. The format also may allow explanations to be completed simultaneously and in parallel with the answer without any performance loss. Thus, an exemplary rule format may be implemented in low-latency applications, such as real-time or quasi-real-time environments, or in low-processing, low-memory hardware.

An exemplary embodiment may implement an exemplary rule format using input from a combination of a digital-analog hybrid system, optical system, quantum entangled system, bio-electrical interface, bio-mechanical interface or suitable alternative in the conditional, “If” part of the rules and/or a combination of the Localization Trigger, Answer Context, Explanation Context or Justification Context. In such an exemplary embodiment, the IF part of the rules may be partially determined, for example, via input from an optical interferometer, a digital-analog photonic processor, an entangled-photon source, or a neural interface. Such an exemplary embodiment may have various practical applications, including medical applications, microscopy applications and advanced physical inspection machines.

An exemplary embodiment may implement an exemplary rule format using a combination of workflows, process flows, process description, state-transition charts, Petri networks, electronic circuits, logic gates, optical circuits, digital-analog hybrid circuits, bio-mechanical interface, bio-electrical interface, quantum circuits or suitable implementation methods.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art (for example, features associated with certain configurations of the invention may instead be associated with any other configurations of the invention, as desired).

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A method for encoding and transmitting knowledge, comprising:
partitioning a set of data to form a plurality of partitions based on a plurality of features found in the data, wherein each partition includes data with related features, comprising:
determining a localization trigger for each partition;
fitting one or more local models to one or more partitions, wherein a local model in the one or more local models corresponds to each partition in the one or more partitions, wherein fitting one or more local models to the one or more partitions comprises providing a local partition input to each partition in the one or more partitions and receiving a local partition output for said partition in the one or more partitions;
determining, for each partition, an equation specific to said partition, wherein each equation comprises one or more coefficients, wherein each coefficient corresponds to one or more of: a level of importance of each feature, a boundary of a feature, a boundary of a partition, possible feature values, feature discontinuity boundaries, feature continuity characteristics, and a transformed feature value, and wherein each equation is configured to produce an answer given a corresponding input based on a set of relevant coefficients among the plurality of coefficients;
determining an explanation relating to each partition, the explanation comprising information corresponding to the set of relevant coefficients;
identifying one or more rules for each partition, each rule comprising the localization trigger and the equation; and
generating explanations associated with each rule.

2. The method for encoding and transmitting knowledge of claim 1, wherein one or more partitions overlap, wherein the method further comprises selecting, with a priority function, one specific partition in the one or more partitions to use as the partition when the one or more partitions overlap.

3. The method for encoding and transmitting knowledge of claim 1, further comprising presenting the answer in the form of at least one of a probability and a predicted value.

4. The method for encoding and transmitting knowledge of claim 1, further comprising presenting the answer in a binary form along with a probability of accuracy.

5. The method for encoding and transmitting knowledge of claim 1, further comprising presenting the explanation in a human-understandable form.

6. The method for encoding and transmitting knowledge of claim 1, further comprising producing one or more additional explanations corresponding to one answer.

7. The method for encoding and transmitting knowledge of claim 1, further comprising identifying a target user for which the answer and the explanation is intended and personalizing the answer and the explanation based on the identification of the target user and one or more external factors, wherein the external factors include data from one or more of: goal-task-action-plan models, question-answering and interactive systems, Reinforcement Learning world models, user/agent models, and workflow systems.

8. The method for encoding and transmitting knowledge of claim 1, further comprising identifying an answer context and an explanation context, by identifying and recording one or more external factors affecting at least one of the answer and the explanation.

9. The method for encoding and transmitting knowledge of claim 1, further comprising structuring the rules in a hierarchy.

10. The method for encoding and transmitting knowledge of claim 1, further comprising encoding the answer and explanation in a machine-readable form.

11. The method for encoding and transmitting knowledge of claim 1, further comprising applying one or more transformations, forming a transformation function pipeline, wherein the transformation function pipeline comprises one or more linear and non-linear transformations, wherein the transformations are applied to the one or more local models.

12. The method for encoding and transmitting knowledge of claim 1, further comprising receiving user feedback and iteratively determining additional applicable rules based on the user feedback, adding the additional rules to a set of rules comprising the one or more rules, and generating explanations associated with the additional rules.

13. A system for encoding and transmitting knowledge, the system comprising a processor and a memory and configured to implement steps of:
partitioning a set of data to form a plurality of partitions based on a plurality of features found in the data, wherein each partition includes data with related features, comprising:
determining a localization trigger for each partition;
fitting one or more local models to one or more partitions, wherein a local model in the one or more local models corresponds to each partition in the one or more partitions, wherein fitting one or more local models to the one or more partitions comprises providing a local partition input to each partition in the one or more partitions and receiving a local partition output for said partition in the one or more partitions;
determining, for each partition, an equation specific to said partition, wherein each equation comprises one or more coefficients, wherein each coefficient corresponds to one or more of: a level of importance of each feature, a boundary of a feature, a boundary of a partition, possible feature values, feature discontinuity boundaries, feature continuity characteristics, and a transformed feature value, and wherein each equation is configured to produce an answer given a corresponding input based on a set of relevant coefficients among the plurality of coefficients;
determining an explanation relating to each partition, the explanation comprising information corresponding to the set of relevant coefficients;
identifying one or more rules for each partition, each rule comprising the localization trigger and the equation; and
generating explanations associated with each rule.

14. The system for encoding and transmitting knowledge of claim 13, further comprising identifying a target user for which the answer and the explanation is intended and personalizing the answer and the explanation based on the identification of the target user.

15. The system for encoding and transmitting knowledge of claim 13, further comprising identifying an answer context and an explanation context, by identifying and recording one or more external factors affecting at least one of the answer, justification, and the explanation.

16. The system for encoding and transmitting knowledge of claim 13, further comprising receiving user feedback and iteratively determining additional applicable rules based on the user feedback, adding the additional rules to a set of rules comprising the one or more rules, and generating explanations associated with the additional rules.

17. A non-transitory computer-readable medium containing program code that, when executed, causes a processor to perform steps of:
partitioning a set of data to form a plurality of partitions based on a plurality of features found in the data, wherein each partition includes data with related features, comprising:
determining a localization trigger for each partition;
fitting one or more local models to one or more partitions, wherein a local model in the one or more local models corresponds to each partition in the one or more partitions, wherein fitting one or more local models to the one or more partitions comprises providing a local partition input to each partition in the one or more partitions and receiving a local partition output for said partition in the one or more partitions;
determining, for each partition, an equation specific to said partition, wherein each equation comprises one or more coefficients, wherein each coefficient corresponds to one or more of: a level of importance of each feature, a boundary of a feature, a boundary of a partition, possible feature values, feature discontinuity boundaries, feature continuity characteristics, and a transformed feature value, and wherein each equation is configured to produce an answer given a corresponding input based on a set of relevant coefficients among the plurality of coefficients;
determining an explanation relating to each partition, the explanation comprising information corresponding to the set of relevant coefficients;
identifying one or more rules for each partition, each rule comprising the localization trigger and the equation; and
generating explanations associated with each rule.

18. The non-transitory computer-readable medium containing program code of claim 17, further comprising encoding the answer in a machine-readable form and presenting the explanation in a human-understandable form.

19. The non-transitory computer-readable medium containing program code of claim 17, further comprising presenting the answer in the form of at least one of: an enumerated value, a classification, a probability, a binary value with a probability of accuracy, a regressed value, a predicted value with a probability of accuracy, an ordered sequence of predicted values, an ordered sequence of regressed values, an ordered sequence of enumerated values, and an ordered sequence of classifications.

20. The non-transitory computer-readable medium containing program code of claim 17, further comprising receiving user feedback and iteratively determining additional applicable rules based on the user feedback, adding the additional rules to a set of rules comprising the one or more rules, and generating explanations associated with the additional rules.

21. The method for encoding and transmitting knowledge of claim 1, wherein the rules are represented in one of: if-then format, a disjunctive normal form, conjunctive normal form, first-order logic assertions, Boolean logic, first order logic, second order logic, propositional logic, predicate logic, modal logic, probabilistic logic, many-valued logic, fuzzy logic, intuitionistic logic, non-monotonic logic, non-reflexive logic, quantum logic, and paraconsistent logic.

22. The method for encoding and transmitting knowledge of claim 1, wherein the method is implemented as one or more of an explainable neural network (XNN), explainable transducer transformer (XTT), explainable spiking network (XSN), explainable memory network (XMN), explainable reinforcement learning agent (XRL), explainable generative adversarial network (XGAN), or an explainable autoencoder/decoder (XAED).

23. The method for encoding and transmitting knowledge of claim 1, wherein an aggregation function merges results from multiple partitions.

24. The method for encoding and transmitting knowledge of claim 1, wherein a split function splits at least one partition into two or more partitions.

25. The method for encoding and transmitting knowledge of claim 11, wherein the transformation pipeline is further configured to perform transformations that analyze one or more temporally ordered data sequences according to the value of one or more variables.

26. The system for encoding and transmitting knowledge of claim 13, wherein the system is implemented on one or more of a field programmable gate array (FPGA), application specific integrated circuit (ASIC), a neuromorphic computing architecture, and a quantum computing architecture.

27. The method for encoding and transmitting knowledge of claim 1, wherein the localization trigger is based on a causal model and further comprises a plurality of conditions on a plurality of attributes with causal variables taken from one or more of a structural causal model or a causal directed acyclic graph.

28. The method for encoding and transmitting knowledge of claim 11, wherein the transformation transforms the prediction output to be structured as one of: a hierarchical tree or network, a causal diagram, a directed or undirected graph, a multimedia structure, and a set of hyperlinked graphs.

29. The method for encoding and transmitting knowledge of claim 1, wherein the explanation indicates the presence of one or more of: bias, strength, weakness, and level of confidence.

30. The method for encoding and transmitting knowledge of claim 1, further comprising a causal analysis.

31. The method for encoding and transmitting knowledge of claim 1, further comprising converting the resulting set of rules into an explainable neural network.

32. The method for encoding and transmitting knowledge of claim 1, further comprising integrating the set of rules with an expert system.

33. The method for encoding and transmitting knowledge of claim 1, wherein the method is implemented as one or more of workflows, process flows, process description, state-transition charts, Petri networks, electronic circuits, logic gates, optical circuits, digital-analogue hybrid circuits, bio-mechanical interface, bio-electrical interface, or quantum circuits.

34. The method for encoding and transmitting knowledge of claim 1, further comprising integrating the set of rules with a digital-analogue hybrid system, optical system, quantum entangled system, bio-electrical interface, bio-mechanical interface, entangled photon source, photonic processor, interferometer, or neural interface.
